(19) United States
(12) Patent Application Publication
(10) Pub. No.: US 2017/0290752 A1
(43) Pub. Date: Oct. 12, 2017

(54) COMPOSITIONS AND METHODS FOR SHAVING

(71) Applicant: Shave Away, LLC, DeMotte, IN (US)

(72) Inventor: Gilbert R. Broom, DeMotte, IN (US)

(21) Appl. No.: 15/094,492

(22) Filed: Apr. 8, 2016

(51) Int. Cl. A61K 8/34; A61K 8/37; A61K 8/86; B26B 21/40; A61K 8/35; A61K 8/36; A61K 8/31; A61K 8/92; A61Q 9/02; A61K 8/97

(52) U.S. Cl. CPC A61K 8/34; A61Q 9/02; A61K 8/37; A61K 8/86; A61K 8/97; A61K 8/35; A61K 8/361; A61K 8/345; A61K 8/31; A61K 8/342; A61K 8/375; A61K 8/922; B26B 21/4081; A61K 2800/591

(57) ABSTRACT

Improved compositions for use during shaving of hair are disclosed as well as methods that employ such improved compositions for shaving. These improved compositions and methods may help to reduce irritation of skin during shaving, reduce irritation of skin after shaving, increase the lifetime of razors used for shaving and/or decrease the quantity of composition needed for each act of shaving.

### BACKGROUND

Humans, throughout the course of time, have removed hair from their bodies for a variety of reasons. For example, removal of hair may be done for a cosmetic purpose—for example, men commonly remove facial hair, and/or women commonly remove hair on legs. Removal of hair may be done for functional purposes—for example, swimmers commonly remove hair from their body to reduce drag in water during races.

Regardless of the reason for removing hair from the skin, the skin—a sometimes delicate organ—often can become irritated during and/or after the removal of hair from the skin.

In light of this background, there remains a need for improved compositions used during the act of shaving, improved methods for preparing compositions used during the act of shaving, and/or improved methods for shaving.

### SUMMARY

In certain aspects, the present disclosure relates to improved compositions, improved methods for preparing compositions, and/or improved methods for shaving hair. In certain preferred aspects, the improved compositions for shaving include SD alcohol 40-B, isopropyl myristate, polysorbate 20, camellia sinesis leaf extract, benzophenone-1, steric acid, propylene glycol, mineral oil, cetyl alcohol, glycerol stearate, S.E. Aloe barbadensis leaf extract, and/or shea butter.

In further aspects, the improved compositions may include one or more colorants, and/or one or more fragrances. In certain preferred embodiments, the colorants include, but are not limited to Green 5, Yellow 10, and/or Orange 4.

In certain aspects, the improved compositions are a liquid phase of matter. In other embodiments, the composition is not a foam, gel, and/or solid phase of matter.

In other aspects, the improved compositions and/or methods of preparing the improved compositions of the present disclosure may be formulated by mixing one or more pre-formulated personal care products and/or compositions. For example, in one embodiment, a pre-formulated composition suitable for use in embodiments disclosed herein may comprise SD Alcohol 40-B, isopropyl myristate, polysorbate 20, camellia sinensis leaf extract, and/or benzophenone-1, and/or another pre- formulated composition may comprise steric acid, propylene glycol, mineral oil, cetyl alcohol, glycerol stearate, S.E. Aloe barbadensis leaf extract, and/or shea butter. In another aspect, a pre-formulated composition suitable for use in embodiments disclosed herein may comprise glycerin, propylene glycol, sodium polyacrylate, triethanolamine, disodium edta, ethyl alcohol, DMDM, aloe vera, and/or vitamin E.

In certain aspects, mixing is affected by the act of shaking the one or more compositions in a vessel. In still other aspects, a vessel may be vented to remove any pressure and/or vacuum that may form during mixing and/or shaking. In still other aspects, compositions disclosed herein may be used in improved methods for removing hair from a body and/or shaving.

In additional embodiments, improved methods for shaving the skin of a body are disclosed. In one embodiment, an improved method for shaving the skin of a body may comprise the acts of applying water to the skin of a body, applying a composition to a body, and/or cutting hair from the skin of a body. In another embodiment, soap may be applied to the skin of a body before and/or after shaving. In other aspects, the water applied to the skin of a body may be cold water, warm water, and/or hot water. In still further embodiments, the skin of the body from which hair is to be removed is located on the face, scalp, arms, legs, and/or the pubic area of a body.

In other embodiments, a small quantity of a composition is applied to the skin of a body. For example, in certain embodiments, the quantity of a composition applied to the skin of a body is about 0.01 fluid ounces, and/or between about 0.01 fluid ounces and about 0.1 fluid ounces. The composition may be applied to the body by spraying a composition to the hands, then transferring the composition to the area of the body to be shaved. In some embodiments, a composition may be applied to the hands or to the area to be shaved by spraying the composition.

Additional embodiments of the invention, as well as features and advantages thereof, will be apparent from the descriptions herein.

### DETAILED DESCRIPTION

For the purpose of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates.

It has been found that improved compositions for use during the removal of hair from skin, sometimes referred to herein as shaving, may be obtained by utilizing various pre-formulated consumer products. These improved compositions may have desirable properties such as low cost, ease of re-formulation, and/or ease of preparation as well as desirable properties during and/or after use.

For example, it has been found that the improved compositions disclosed herein may reduce clogging of multi-razor safety razors, may reduce the number of nicks when shaving, and/or may reduce the irritation of the skin after shaving.

It has also been found that the improved compositions disclosed herein may be used during shaving as well as for uses after shaving, such as for example, but not limited to an aftershave. Pre-formulated compositions that may be utilized in embodiments of the present invention include liquid pre-shave compositions and/or lotion products. Such liquid pre-shave compositions include, but are not limited to pre-shave liquids designed for use with electric razors. In one preferred embodiment, a pre-formulated compositions marketed by Best Choice brand called “Electric Razor Pre- Shave with Soothing Green Tea,” and distributed by Valu Merchandisers, Co., 5000 Kansas Ave., Kansas City, Kans. 66106 may be used. This composition may comprise SD Alcohol 40-B, isopropyl myristate, water, polysorbate 20, fragrance, camellia sinensis leaf extract, and/or benzophenone-1.

Another pre-formulated composition that may be used in various embodiments of the present disclosure includes, but is not limited to “Nature's Advantage” lotion distributed by Marianna Industries, Omaha, Neb., 66137, which may comprise steric acid, propylene glycol, mineral oil, cetyl alcohol, glycerol stearate, S.E. Aloe barbadensis leaf extract, and/or Vitellaria paradoxa (shea butter).

Other ingredients may be utilized in compositions of the present disclosure. Such other ingredients include, but are not limited to water, solvents, fragrances, dyes and/or colorants, and/or other ingredients which may impart a desired effect on the composition.

For example, additional solvents that may be included in embodiments of the present disclosure include, but are not limited to, alcohols. For example, methanol, ethanol, and/or propanols such as isopropanol and/or n-propanol, may be utilized in certain embodiments.

Fragrances, for example, those compounds which have a pleasant odor may be utilized in various embodiments of the present invention. Such fragrances may include synthetic fragrances, as well as those fragrances obtained from natural sources such as essential oils or extracts of flowers may be used.

Colorants, or those compositions which impart a desired color to a compositions may be added. Such colorants include, but are not limited to, Green 5, Yellow 10, and/or Orange 4, as well as other colorants known in the art.

As will be understood by one of ordinary skill in the art to which the present disclosure relates, various quantities, ratios, and/or techniques may be utilized in the practice of the present disclosure.

In order to promote a further understanding of the present invention and its various embodiments, the following specific examples are provided. It will be understood that these examples are illustrative and not limiting of the invention.

### EXAMPLE 1

### Preparation of Composition

### Materials and Methods

To an empty one quart vessel was added about 10 fluid ounces (about 1 part) of a composition of matter comprising SD Alcohol 40-B, isopropyl myristate, water, polysorbate 20, fragrance, camellia sinensis leaf extract, benzophenone-1 was obtained (Best Choice brand Electric Razor Pre-Shave with Soothing Green Tea, distributed by Valu Merchandisers, Co., 5000 Kansas Ave., Kansas City, KS 66106). About 20 fluid ounces (about 2 parts) of a composition comprising water, steric acid, propylene glycol, mineral oil, cetyl alcohol, glycerol stearate, S.E. Aloe barbadensis leaf extract, and Vitellaria paradoxa (shea butter) was added. One such composition is marketed and distributed under the name “Nature's Advantage” (Marianna Industries, Omaha, Neb., 66137). The vessel was then sealed, and the resulting composition shaken vigorously.

During and/or after shaking, it was noticed that the vessel and composition were warm to the touch. Any pressure that developed during shaking was released by venting the vessel.

The resulting composition was transferred to a manual spray bottle.

Other pre-formulated compositions were tested for incorporation into the compositions of the present disclosure. For example, St. Ives Swiss formulation, St. Ives aloe vera advanced formulation, Jergens original scent formulation, Jergens cherry almond formulation, Olay body lotion, Vaseline Intensive Care herbal formulation, and/or Vaseline Intensive Care aloe vera formulation were tested. These pre-formulated compositions did not appear to work according methods of the present disclosure. Without being bound by theory, phase separation was commonly observed when these certain pre-formulated compositions were used.

Various ratios and/or quantities of pre-formulated compositions were also tested for use in compositions and methods of the present disclosure. For example, ratios of about 1 to about 1, of about 1 to about 3, of about 1 to about 4 were tested. However, compositions made from these ratios of pre-formulated compositions had properties that made them less desirable. For example, compositions made from these ratios of pre-formulated compositions sometimes separated into two or more phases.

### EXAMPLE 2

### Preparation of Composition

### Materials and Methods

4 fluid ounces of the composition of EXAMPLE 1 was removed from the vessel and discarded. 4 fluid ounces of a composition comprising glycerin, propylene glycol, sodium polyacrylate, triethanolamine, disodium EDTA, ethyl alcohol, DMDM, aloe vera, vitamin E, and water was then added. Such a composition is marketed and distributed under the name “St. Eden Spa” baby oil gel (distributed by Core Brands, 1301 Wicomico St., Baltimore, Md. 21230). The vessel was sealed, and the resulting compositions was shaken vigorously. The resulting composition was transferred to a manual spray bottle.

### EXAMPLE 3

### Method for Shaving with Composition of Example 1 or Example 2

### Materials and Methods

The composition of matter from Example 1 or Example 2 was used for shaving by wetting the area to be shaved, washing the area to be shaved with soap, dabbing dry the area to be shaved, applying the composition from Example 1 or Example 2 by spraying two sprays into the hands and applying the composition to the area to be shaved, spraying two additional sprays in to the hands and applying the composition to the area to be shaved, and/or shaving the area to be shaved with a commercial multi-blade safety razor.

### Results

It was observed that by using the composition from Example 1 or Example 2, that the number of nicks in the area to be shaved was reduced, the razor blade used exhibited fewer clogs from the hair removed from the area to be shaved, and the lifetime of the safety razor blade used was increased. For example, a razor blade that would typically last for one month lasted for 11 months when the compositions from Example 1 or Example 2 was used.

In addition to the above-mentioned benefits, it was also observed that severed hairs were less likely to clog the interstices between multi-blade safety razors during and after shaving when using the compositions of Example 1 and/or Example 2, and/or multi-blade safety razors lasted for an increased number of shaves.

EXAMPLE 4

### Alternative Method for Shaving with Composition of Example 1 or Example 2

### Materials and Methods

After shaving according to the procedures listed in Example 3, 2 additional sprays of the composition of Example 1 or Example 2 can be made to the hands, and the composition then applied to the area to be shaved.

### Results

It was observed that certain testers enjoyed additional benefits when the compositions of Example 1 or Example 2 were applied to the area to be shaved after shaving. For example, some testers observed reduced irritation of the area to be shaved when the composition from Example 1 or Example 2 was applied after shaving.

The uses of the terms “a” and “an” and “the” and similar references in the context of describing the invention (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

1. An improved composition for shaving, comprising:
SD Alcohol 40-B, isopropyl myristate, polysorbate 20, camellia sinensis leaf extract, benzophenone-1, steric acid, propylene glycol, mineral oil, cetyl alcohol, glycerol stearate, S.E. Aloe barbadensis leaf extract, and shea butter.

2. The composition of claim 1, further comprising one or more fragrances.

3. The composition of claim 1, further comprising one or more colorants.

4. The composition of claim 3, wherein said one or more colorants are Green 5, Yellow 10, or Orange 4.

5. The composition of claim 1, wherein said composition is a liquid.

6. The composition of claim 1, wherein said composition is not a foam, gel, or solid.

7. A method for preparing an improved composition for shaving, comprising the acts of:
providing a first composition, comprising SD Alcohol 40-B, isopropyl myristate, polysorbate 20, camellia sinensis leaf extract, benzophenone-1;
providing a second composition, comprising steric acid, propylene glycol, mineral oil, cetyl alcohol, glycerol stearate, S.E. Aloe barbadensis leaf extract, and shea butter.
combining said first composition and said second composition in a vessel.

8. The method according to claim 7, further comprising the act of shaking said vessel comprising said first composition and said second composition.

9. The method according to claim 7, further comprising the act of transferring some or all of the composition to a spray bottle.

10. The method according to claim 7, further comprising the act of combining a third composition with said first composition and said second composition, wherein said third composition comprises glycerin, propylene glycol, sodium polyacrylate, triethanolamine, disodium edta, ethyl alcohol, DMDM, aloe vera, and vitamin E.

11. An improved method for removing hair from a body, comprising the acts of:
applying the composition of claim 1 to the skin of said body;
applying water to said body;
cutting said hair with a razor blade.

12. The method of claim 11, further comprising the act of applying the composition of claim 1 to said body after the act of cutting said hair with a razor blade.

13. The method of claim 11, further comprising the act of applying soap to said area to be shaved.

14. The method of claim 11, wherein the water used during said applying water to said body step is cold water, warm water, or hot water.

15. The method of claim 11, wherein the skin of said body is located on the face, scalp, arms, legs, or pubic area of said body.

16. The method of claim 11, wherein the quantity of said composition of claims 1 applied to the skin of said body is about 0.01 fluid ounces.

17. The method of claim 11, wherein the quantity of said composition of claim 1 applied to the skin of said body is between about 0.01 fluid ounces and about 0.1 fluid ounces.
